(19) United States
(12) Patent Application Publication
(10) Pub. No.: US 2011/0160780 A1
(43) Pub. Date: Jun. 30, 2011

(54) Bone breaking instrument

(76) Inventor: Jason M. Yamada, Rolling Hills Estates, CA (US)

(21) Appl. No.: 12/931,901

(22) Filed: Feb. 14, 2011

Related U.S. Application Data
(63) Continuation of application No. 11/895,810, filed on Aug. 28, 2007.
(60) Provisional application No. 60/882,940, filed on Dec. 31, 2006.

(51) Int. Cl. A61B 17/56

(52) U.S. Cl. 606/86 R

(57) ABSTRACT

In an internal sinus manipulation procedure for augmenting bone of a dental patient between a bony floor of the patient's sinus and a raised portion of the patient's sinus membrane, a process for removing bone fragments extending laterally into an upward channel adjacent its upper end comprising selecting a bone breaking instrument including longitudinally elongated handle and a laterally extending head secured to an upper end of the handle and including a substantially flat lower surface that is inclined upwardly and laterally toward the handle at an angle of less that 90 degrees, inserting the selected instrument upward into the channel with the head of the instrument above a bone fragment and with its flat lower surface hooking over the fragment and pulling down on the handle to break off the bone fragment.

### RELATED PATENT APPLICATIONS

The present application is a Continuation of U.S. patent application Ser. No. 11/895,810 filed Aug. 28, 2007 and claims benefit of U.S. Provisional Patent application Ser. No. 60/882,940 filed Dec. 31, 2006, which is herein incorporated by reference. The present application also relates to the subject matter of U.S. patent application Ser. No. 11/895,823, also filed Aug. 28, 2007, entitled “Internal Sinus Manipulation (ISM) Procedure For Facilitating Sinus Floor Augmentation In Dental Procedures”, now U.S. Pat. No. 7,622,188, issued Feb. 16, 2010, which is incorporated herein by this reference.

### BACKGROUND OF INVENTION

As stated in the above-identified U.S. Pat. No. 7,622,188, during the described procedure and in the formation of an upward channel in the bone leading to the sinus floor of a patient, small inwardly directed bone fragments may surround or extend into the upper opening of the sinus bone channel. As illustrated in FIG. 4 of the above-identified United States patent, after the formation of a small pocket between he floor of the sinus and the sinus membrane, such bone fragments are removed, preferably using a bone breaking instrument. The present invention is directed to a preferred form of such a sinus bone-breaking instrument.

### SUMMARY OF INVENTION

Basically, the sinus bone-breaking instrument of the present invention comprises a longitudinally extending handle and a relatively small laterally extending distal head designed to fit upward into a bone channel. To enable the distal head to effectively remove bone fragments extending into the bone channel, the distal head includes a laterally inward and upward inclined lower surface. The lower inclined surface is employed to hook the bone fragments and with a downward pulling on the handle to break off the bone fragments leaving a clear opening to the sinus membrane.

### BRIEF DESCRIPTION OF ACCOMPANYING DRAWINGS

FIG. 1 is a perspective view of the preferred bone-breaking instrument.

FIG. 2 is an enlarged fragmentary sectional side view of an upper portion of the instrument of FIG. 1 showing the laterally extending distal head of the instrument having an inwardly and upwardly inclined lower bone-hooking surface.

FIG. 3 is a slightly enlarged fragmentary sectional front view of the distal head of the instrument depicted in FIG. 2.

### DETAILED DESCRIPTION OF INVENTION

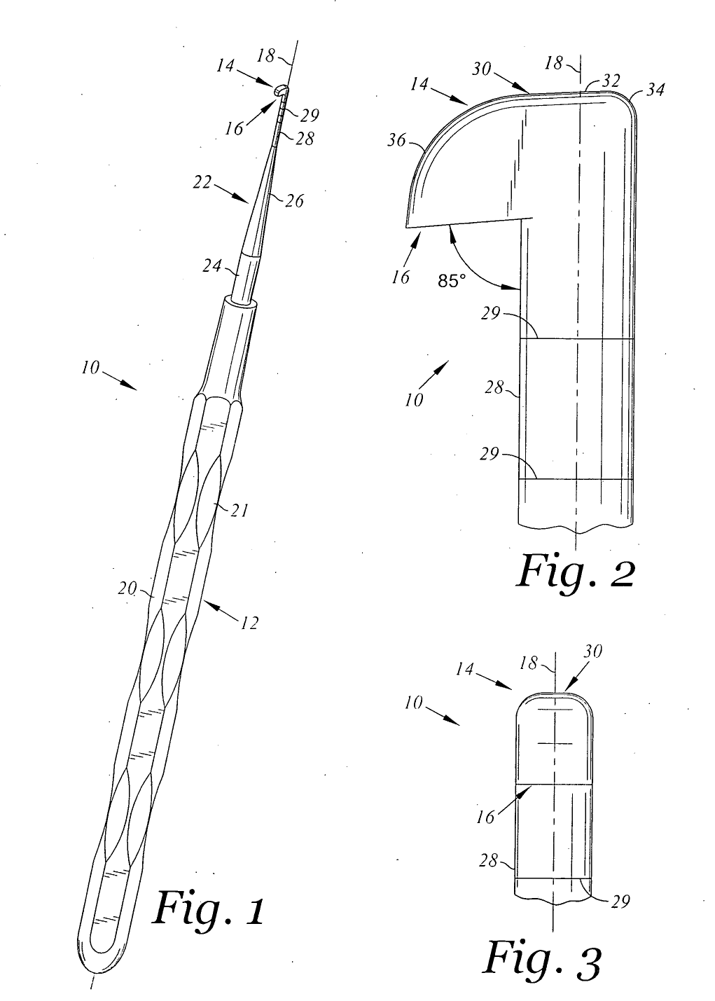

As depicted in FIG. 1, the bone-breaking instrument 10 of the present invention comprises a longitudinally extending handle 12 and a laterally extending distal head 14 with an inwardly and upwardly inclined lower surface 16.

As shown in FIG. 1, the handle 12 is longitudinally elongated on a longitudinal axis 18 of the instrument 10 and includes a slightly enlarged lower hand-holdable portion 20 having a longitudinally fluted outer surface 21 for enhancing the grip-ability of the instrument 10.

As illustrated, the distal head 14 is secured to the handle by an intermediate longitudinally extending portion 22 of the instrument 10 comprising a cylindrical lower section 24, an axially elongated frusto-conical middle section 26 and an upper cylindrical section 28 of reduced diameter compared to the diameter of the lower section 24. As depicted in FIG. 1, the cylindrical lower section 24 is secured to and is of a slightly reduced diameter relative an upper end of the hand-holdable portion 20 of the handle 12. The bottom of the frusto-conical middle section 26 is secured to a top of the lower section 24, is axially elongated on the axis 18 and is reduced in lateral diameter as to approaches its relatively small upper end joined to a lower end of the upper cylindrical section 28. The upper cylindrical section 28, in turn, extends, longitudinally on the axis 18 upward to support the distal head 14 that is secured to and supported by the end of the cylindrical section 28, as best shown in FIG. 2. As shown in FIGS. 1 and 2, the outer surface of the upper cylindrical section 28 below the distal head 14 carries a plurality of horizontal vertically and evenly spaced depth markers 29 for indicating the user of the instrument 10 the depth of the instrument in a bone channel prior to use in the breaking of bone fragments. By way of example, the vertical spacing of the depth markers 29 may be about 2 millimeters.

As most clearly shown in FIGS. 2 and 3, the distal head 14 extends upwardly from the upper end of the upper cylindrical section 28 and laterally from the longitudinal axis 18 of the instrument 10. An upper surface 30 of the distal head 14 is substantially flat and perpendicular to the axis 18 of the instrument 10 in its central region 32 and curved downwardly at its rearmost end 34 and at its forwardmost end 36 terminating at the lower surface 16. As illustrated in FIG. 2, the lower surface 16 is substantially flat and laterally and upwardly inclined toward the longitudinal axis 18 at an angle of less than 90 degrees to the longitudinal axis, preferably about 85 degrees as indicated. As illustrated in FIG. 3, the upper surface 30 of the distal head 14 is also curved downwardly at right and left side of the head 14 (as depicted in FIG. 3).

Thus configured, the instrument 10 is shaped for comfortable hand gripping with the handle 12, intermediate 22 and distal head 14 portions extending upwardly and into a bone channel to break off bone fragments extending inwardly from the wall of the channel. The lower inclined surface 16 of the distal head 14 is employed to hook the bone fragments and with a downward pulling on the handle 12 to break off the bone fragments leaving a clear opening in the bone channel to the sinus membrane.

While a particular preferred embodiment of the bone-breaking instrument has been illustrated and described above, it is appreciated that changes and modifications may be made in the illustrated embodiment without departing from the spirit of the invention. Accordingly, the scope of present invention is to be limited only by the terms of the following claims.

1. In an internal sinus manipulation procedure for augmenting bone of a dental patient between a bony floor of the patient's sinus and a raised portion of the patient's sinus membrane that comprises creating an upward channel in bone within a maxillary posterior area of the patient such that an upper end of the channel extends to a base of a bony floor of the sinus and is sized to expose a portion of the patient's sinus membrane over the upper end of the upward channel, the process of removing fragments of bone extending laterally into the upward channel adjacent its upper, comprising:
selecting a bone breaking instrument including (i) a handle that is longitudinally elongated on an axis of the instrument and (ii) a laterally extending head secured to an upper end of the handle and including a substantially flat lower surface that is inclined upwardly and laterally toward the handle at an angle of less that 90 degrees,
inserting the selected bone breaking instrument upwardly into the upward channel,
positioning the head of the instrument above a bone fragment with its flat lower surface hooking over the fragment and
pulling down on the handle of the instrument to break off the bone fragment.

2. The bone fragment removal procedure of claim 1 wherein the head of the selected bone breaking instrument further comprises:
an upper surface that is substantially flat and substantially perpendicular to the axis of the instrument in a central region thereof and that curves downward at a rear end and at a forward end terminating at the lower flat surface of the head.
